(19) United States
(12) Patent Application Publication
(10) Pub. No.: US 2010/0057178 A1
(43) Pub. Date: Mar. 4, 2010

(54) METHODS AND APPARATUS FOR SPINAL CORD STIMULATION USING EXPANDABLE ELECTRODE

(75) Inventor: Bruce J. Simon, Mountain Lakes, NJ (US)

Correspondence Address: ELECTROCORE INC., 51 GILBRALTAR DRIVE, SUITE 2F, POWER MILL PLAZA, MORRIS PLAINS, NJ, 07950-1254, US

(73) Assignee: ElectroCore, Inc., Morris Plains, NJ (US)

(21) Appl. No.: 12/557,936

(22) Filed: Sep. 11, 2009

Related U.S. Application Data
(63) Continuation-in-part of application No. 12/394,972, filed on Feb. 27, 2009.
Application No. 12/394,972 is a continuation-in-part of application No. 12/338,191, filed on Dec. 18, 2008.
Application No. 12/338,191 is a continuation-in-part of application No. 12/246,605, filed on Oct. 7, 2008.
Application No. 12/246,605 is a continuation-in-part of application No. 11/735,709, filed on Apr. 16, 2007.
(60) Provisional application No. 61/043,802, filed on Apr. 10, 2008.
(60) Provisional application No. 61/043,805, filed on Apr. 10, 2008.
(60) Provisional application No. 60/978,240, filed on Oct. 8, 2007.
(60) Provisional application No. 60/792,823, filed on Apr. 18, 2006.

(51) Int. Cl. A61N 1/00

(52) U.S. Cl. 607/117

(57) ABSTRACT

The present invention provides systems, apparatus and methods for selectively applying electrical energy to body tissue. More specifically, systems and methods are provided for introducing a spinal cord stimulation electrode device into a patient's epidural space through a small portal, such as a percutaneous penetration, and then expanding the electrode device once inside the epidural space to achieve a larger footprint of contact on the dura. This substantially prevents migration of the electrode within the epidural space and provides for more efficient and effective treatment.

### CROSS REFERENCE TO RELATED APPLICATIONS

The present application is a continuation-in-part of U.S. patent application Ser. No. 12/394,972, filed Feb. 27, 2009, which claims the benefit of provisional patent application Ser. Nos. 61/043,805 and 61/043,802, filed Apr. 10, 2007, and which is a continuation-in-part of U.S. patent application Ser. No. 12/338,191, filed Dec. 18, 2008 the complete disclosures of which are incorporated herein by reference for all purposes.

This application is also a continuation-in-part of U.S. patent application Ser. No. 12/246,605 filed Oct. 7, 2008, which claims the benefit of U.S. Provisional Patent Application No. 60/978,240, filed Oct. 8, 2007 and which is a continuation-in-part of U.S. patent application Ser. No. 11/735,709, filed Apr. 16, 2007 which claims the benefit of U.S. Provisional Patent Application No. 60/792,823, filed Apr. 18, 2006

This application is also related to commonly assigned co-pending U.S. patent Ser. Nos. 11/555,142, 11/555,170, 11/592,095, 11/591,340, 11/591,768, and 11/754,522, the complete disclosures of which are incorporated herein by reference for all purposes.

### BACKGROUND OF THE INVENTION

The present invention relates to the delivery of electrical energy to bodily tissues for therapeutic purposes, and more specifically to devices and methods for treating conditions through delivery of electrical energy using a balloon and electrode device.

The use of electrical stimulation for treatment of medical conditions has been well known in the art for nearly two thousand years. It has been recognized that electrical stimulation of the brain and/or the peripheral nervous system and/or direct stimulation of the malfunctioning tissue, which stimulation is generally a wholly reversible and non-destructive treatment, holds significant promise for the treatment of many ailments.

For many years, electrical stimulation of nervous tissue has been used to control chronic pain or treat other disorders. This therapy originates from an implanted source device, called an electric signal generator. The electrical signals, usually a series of brief duration electrical pulses, are delivered through one or more implanted leads that communicate with the source device, and contain several conductive metal electrodes to act as low impedance pathways for current to pass to tissues of interest. For example, in spinal cord stimulation (SCS) techniques, electrical stimulation is provided to precise parts of the human spinal cord through a lead that is usually deployed in the epidural space dorsal to the spinal cord. Such techniques have proven effective in treating or managing disease and chronic pain conditions.

The use of spinal cord stimulation (SCS) in the management of pain syndromes is a minimally invasive and reversible, implantable neurostimulation modality. This modality has been shown clinically to be effective over a range of maladies including ischemic heart disease—refractory angina pectoris, low back pain with radiculopathy, failed-back surgery syndrome (FBSS), abdominal pain, peripheral vascular disease, and complex regional pain syndrome (CRPS). Reports of SCS clinical success range from 50% to 80% with reductions in medication requirements as well as improvements in pain intensity scores, quality of life (QOL) enhancements, corrected function, and bolstered chances of returning to work.

Spinal cord stimulators typically include one or more electrode leads implanted in the epidural space either percutaneously or by surgical laminectomy or laminotomy. A pulse generator or RF receiver may be implanted, for example in the abdomen or buttocks, to apply an electric impulse to the electrode(s) to block pain signals from reaching the brain such that the patient receives a mild tingling sensation in lieu of the pain.

Percutaneous leads are small diameter leads that may be inserted into the human body through a Tuohy (non-coring) needle, which includes a central lumen through which the lead is guided. Percutaneous leads are advantageous because they may be inserted into the body with a minimum of trauma to surrounding tissue. On the other hand, the designs of lead structures that may be incorporated into percutaneous leads are limited because the lead diameter or cross-section must be small enough to permit the lead to pass through the Tuohy needle, generally less than 2.0 mm diameter. Typically, the electrodes on percutaneous leads are cylindrical metal structures, with a diameter of approximately 1.0 mm and a length of 4.0 to 10.0 mm. Of course, half of each of these electrodes, facing away from the tissue of interest, is not very useful in delivering therapeutic current. Thus the surface area of electrodes that face the tissue to be excited is small, typically 3.0 to 10.0 square mm.

Ideally, an implantable electrode for tissue stimulation in the spinal cord must have several additional features for use in the human body. For one, substantially large conducting electrodes are needed to safely and reliably pass stimulation electrical pulses of adequate amplitudes to excite tissue cells over indefinitely long periods of time. In addition, to minimize surgical trauma during implantation, the electrodes should assume a one dimensional shape that is very narrow inside the lead body (or sheath) for passage through a small catheter or Tuohy needle, and have the ability to assume a two dimensional shape when outside the lead body. Since there may be considerable deposits of fibrosis or scar tissue around each electrode within a few months of permanent implantation, if necessary, the lead should be able to be removed by gentle traction on the lead body, and have all parts easily disengage from the tissue.

### SUMMARY OF THE INVENTION

The present invention provides systems, apparatus and methods for selectively applying electrical energy to body tissue. More specifically, systems and methods are provided for introducing a spinal cord stimulation electrode device into a patient's epidural space through a small portal, such as a percutaneous penetration, and then expanding the electrode device once inside the epidural space to achieve a larger footprint of contact on the dura. This substantially prevents migration of the electrode within the epidural space and provides for more efficient and effective treatment.

In one aspect of the invention, a device for delivering electrical energy to a patient includes an enclosure having a longitudinal axis and an outer wall movable between a collapsed configuration for percutaneous introduction into the patient and an expanded configuration wherein the outer wall extends laterally outward from the longitudinal axis. The outer wall has a larger surface area for contacting tissue in the expanded configuration. An electrode is coupled to at least a portion of the outer wall of the enclosure and to a source of electrical energy for delivering an electrical impulse to the electrode in the expanded configuration.

In the preferred embodiments, the device further includes a fluid delivery system including a fluid passage coupled to the interior of the enclosure for delivering a fluid into the interior of the enclosure. The fluid expands the enclosure laterally outward from the longitudinal axis.

In one specific embodiment, the electrode comprises at least a portion of the outer wall of the enclosure such that the enclosure functions as an electrode balloon. The outer wall preferably expands laterally outward in first and second directions from the longitudinal axis in the expanded configuration to form a substantially planar surface extending in opposite directions from the axis. In this manner, the outer wall forms first and second contact surfaces extending laterally outward from the longitudinal axis in the expanded configuration, wherein the electrode forms part of the first contact surface and the second contact surface comprises an insulating material. The first and second contact surfaces are on opposite sides of the outer wall such that the electrical contact surface contacts the dura and the insulating contact surface will mitigate undesirable stimulation of surrounding tissue structures within the epidural space.

The material of the balloon is preferably very soft and flexible, e.g., elastic, such that it gently conforms to the surrounding tissue, which allows the electrical energy to be applied uniformly to the target tissue. In addition, one skilled in the art will recognize that this configuration allows the balloon to conform to a variety of different tissue shapes and structures within the patient's body. Another advantage of the invention is that the balloon has a larger tissue contact area in the inflated configuration, which allows the device to be applied to a larger tissue treatment area. This can be particularly advantageous when the device is introduced percutaneously into the epidural space for spinal cord stimulation applications.

In an alternative embodiment, the electrode is housed within the enclosure and electrically coupled to the outer wall. The electrode may be spaced from the outer wall and electrically coupled thereto by an electrically conductive fluid delivered into the interior of the balloon. In this configuration, at least a portion of the outer wall will comprise an electrically permeable material for allowing an electrical impulse to pass therethrough to the target site. Alternatively, the electrode may be directly attached to the outer wall. In this embodiment, the fluid need not be conductive.

In one embodiment, the return electrode is a return pad located on a surface of the patient's skin, such as the back or hip, and the electrode within the balloon acts as the tissue treatment or active electrode. In alternative embodiments, the return electrode may be located closer to the active electrode, e.g., within or part of the balloon, coupled to the electrode lead outside of the balloon or within or part of a second balloon. In these embodiments, the electrical energy will not flow completely through the patient's body, i.e., the current will generally flow from the active electrode through the patient's tissue at the target site and to the return electrode.

In an exemplary embodiment, the source of electrical energy is an electrical signal generator operating to apply at least one electrical signal to the electrode such that, when the enclosure is positioned in a spinal cord of the patient, an electro-magnetic field emanates from the electrode to at least one of nerves and muscles in a vicinity of the spinal cord. The electric signal is preferably of a frequency between about 1 Hz to 3000 Hz, a pulse duration of between about 10-1000 us, and an amplitude of between about 1-20 volts.

In another aspect of the invention, a method for treating an ailment in a patient includes the steps of introducing an enclosure through a percutaneous penetration of a patient to a target location adjacent to or near a spine of the patient (e.g., the epidural space) and expanding an outer wall of the enclosure in a lateral direction from its longitudinal axis to contact tissue at the target location. Electrical energy is applied to an electrode coupled to the outer wall of the enclosure to modulate one or more nerves within the spine of the patient. In a preferred embodiment, the enclosure is a balloon that is expanded by delivering a fluid into the interior of the balloon. The outer wall forms a substantially planar contact surface for contacting a dura within the epidural space. The method further comprises insulating the opposite surface of the outer wall to minimize damage to surrounding tissue or unwanted stimulation of surrounding nerves or muscles.

In one embodiment, the method includes applying an electrical impulse to a sympathetic nerve chain of a patient to block, stimulate and/or modulate nerve signals to treat a gastrointestinal disorder of the patient. In this embodiment, an electrical impulse can be applied to increase an intestinal and/or gastric motility of the patient, decrease pain associated with irritable bowel syndrome and/or improve intestinal peristalsis function within the patient.

In an exemplary embodiment, the present invention includes a method of increasing intestinal motility of a patient suffering from post-operative ileus. In this procedure, the balloon electrode of the present invention is introduced through a percutaneous penetration in the patient and advanced to an epidural space between T5 and L2, preferably around T7. Fluid is delivered into the interior of the balloon to inflate the balloon and thereby contact an expanded surface area of the dura as described above. An electrical impulse is applied to the electrode balloon; preferably having a frequency between about 10 Hz to 200 Hz, preferably between about 25 to 50 Hz, a pulse duration of between about 20-400 us, and an amplitude of between about 1-20 volts. The impulse modulates one or more nerves around the epidural space to at least partially improve intestinal peristalsis resulting from the operation.

Other aspects, features, advantages, etc. will become apparent to one skilled in the art when the description of the invention herein is taken in conjunction with the accompanying drawings.

### BRIEF DESCRIPTION OF THE DRAWINGS

For the purposes of illustrating the various aspects of the invention, there are shown in the drawings forms that are presently preferred, it being understood, however, that the invention is not limited by or to the precise arrangements and instrumentalities shown.

FIG. 1 schematically illustrates an exemplary spinal cord stimulation system according to the present invention;

FIG. 2 is a partial cross-sectional view of a distal portion of an electrode lead in a collapsed configuration according to the present invention;

FIG. 3 is a partial cross-sectional view of the electrode lead of FIG. 2 in an expanded configuration;

FIGS. 4A and 4B illustrate an alternative embodiment of the present invention; and

FIG. 5 is a perspective view of the electrode lead of FIGS. 2 and 3 being advanced to a target location within the spinal cord according to the present invention; and

FIG. 6 is a cross-section through the posterior aspect of the thoracic spine with the electrode lead of the present invention in position for applying an electrical impulse to one or more nerves within the epidural space according to the present invention.

### DETAILED DESCRIPTION OF THE PREFERRED EMBODIMENTS

In the present invention, electrical energy is applied to one or more electrodes to deliver an electromagnetic field to a patient. The invention is particularly useful for applying electrical impulses that interact with the signals of one or more nerves, or muscles to achieve a therapeutic result, such as treating ischemic heart disease—refractory angina pectoris, low back pain with radiculopathy, failed-back surgery syndrome (FBSS), abdominal pain, peripheral vascular disease, complex regional pain syndrome, treating ileus conditions, IBS, and/or any other ailment affected by nerve transmissions. For convenience, the remaining disclosure will be directed specifically to the treatment of nerves within the spinal cord with a device introduced through a percutaneous penetration in the patient, but it will be appreciated by those skilled in the art that the systems and methods of the present invention can be applied equally well to other tissues and nerves of the body, including but not limited to other parasympathetic nerves, sympathetic nerves, spinal or cranial nerves, e.g., optic nerve, facial nerves, enteric nerves, vestibulocochlear nerves and the like.

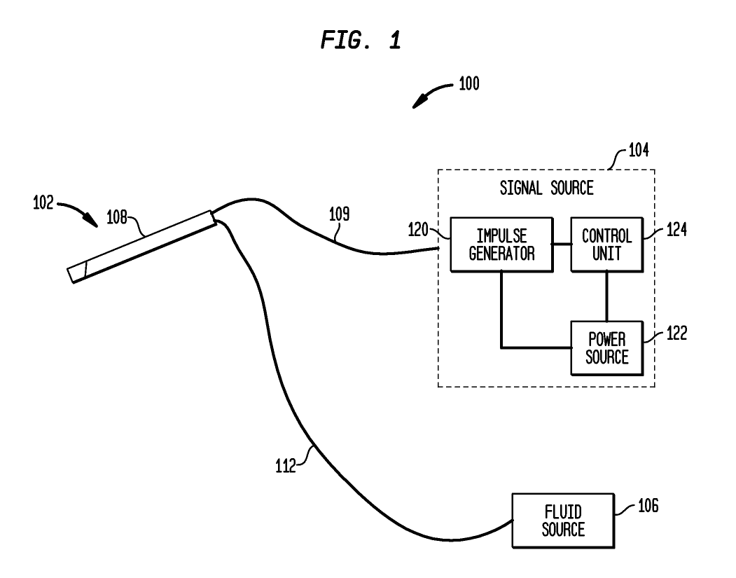

Referring to the drawings in detail, wherein like numerals indicate like elements, FIG. 1 schematically illustrates an exemplary spinal cord stimulation system 100 according to the present invention. System 100 comprises an electrode lead 102 coupled to an electrical signal generator or source 104 for providing an electrical impulse to a target tissue and a fluid source 106 for supplying fluid to the electrode lead 102. Electrode lead 102 includes an elongated shaft or connector 108 which may be flexible or rigid, with flexible shafts optionally including support cannulas or other structures (not shown). Lead shaft 108 is coupled to fluid source 104 via a fluid tube 112 and to electrical source 104 through electrical connector 109. System 100 may also include a return electrode (not shown) adapted for placement on the outer surface of the patient's skin (e.g., the back or buttocks) such that the electrical current passes through the target site and the patient's body to the return electrode. Alternatively, electrode lead 102 may include both the anode and cathode such that the electrical current is confined to the target site. In some embodiments, electrode lead 102 will include multiple electrodes (not shown) that can be alternatively turned on and off to apply the electrical impulse to different parts of the lead 102.

Electrical source 104 operates to apply at least one electrical signal to electrode lead 102 such that, when lead 102 is positioned in the spinal cord of a patient, an electro-magnetic field emanates from the lead 102 to the anatomy of the mammal in the vicinity of the spinal cord to achieve a therapeutic result. Spinal cord stimulators are well known in the art. Examples of conventional electrode leads for such stimulators are the Restore Advanced Neurostimulator sold by Medtronic or the electrodes leads sold by AD-Tech Medical Instrument Corp.

Electrical source 104 may be tailored for the treatment of a particular ailment and may include an electrical impulse generator 120, a power source 122 coupled to the electrical impulse generator 120, and a control unit 124 in communication with the electrical impulse generator 120 and the power source 122. The electrodes provide source and return paths for the at least one electrical signal to/from the electrode lead 102 and the return electrode (which is either located on lead 102 or elsewhere as discussed above). The control unit 124 may control the electrical impulse generator 120 for generation of the signal suitable for amelioration of the ailment when the signal is applied to the electrode lead 102. It is noted that source 104 may be referred to by its function as a pulse generator.

A suitable electrical voltage/current profile for the stimulating, blocking and/or modulating impulse to the portion or portions of one or more nerves and/or muscles may be achieved using the pulse generator 120. In a preferred embodiment, the pulse generator 120 may be implemented using the power source 122 and control unit 124 having, for instance, a processor, a clock, a memory, etc., to produce a pulse train to the electrode(s) that deliver the blocking and/or modulating fields to the nerve resulting from the electrical impulses.

The parameters of the modulation signal are preferably programmable, such as the frequency, amplitude, duty cycle, pulse width, pulse shape, etc. The impulse signal preferably has a frequency, an amplitude, a duty cycle, a pulse width, a pulse shape, etc. selected to influence the therapeutic result, such as stimulating, blocking and/or modulating some or all of one or more nerve transmissions. For example, assuming the aforementioned impedance characteristics of the device 100, the at least one electrical signal may be of a frequency between about 1 Hz to 3000 Hz, a pulse duration of between about 10-1000 us, and an amplitude of between about 1-20 volts. For example, for treating post-operative ileus (discussed below), the electrical signal may be of a frequency between about 25 Hz to 50 Hz. The at least one electrical signal may have a pulsed on-time of between about 50 to 1000 microseconds, such as between about 100 to 300 microseconds, such as about 200 microseconds. The at least one electrical signal may have an amplitude of about 5-15 volts, such as about 12 volts. The at least one electrical signal may include one or more of a full or partial sinusoid, a square wave, a rectangular wave, and triangle wave.

Although the specific implementation of the signal source is not of criticality to the invention, by way of example, the source may be purchased commercially, such as a Model 7432 available from Medtronic, Inc. Alternatively, U.S. Patent Application Publications 2005/0075701 and 2005/0075702, both to Shafer, both of which are incorporated herein by reference, contain descriptions of pulse generators that may be applicable for implementing the signal source of the present invention.

An alternative implementation for the signal source of the present invention may be obtained from the disclosure of U.S. Patent Publication No.: 2005/0216062, the entire disclosure of which is incorporated herein by reference. U.S. Patent Publication No.: 2005/0216062 discloses a multi-functional electrical stimulation (ES) system adapted to yield output signals for effecting faradic, electromagnetic or other forms of electrical stimulation for a broad spectrum of different biological and biomedical applications. The system includes an ES signal stage having a selector coupled to a plurality of different signal generators, each producing a signal having a distinct shape such as a sine, a square or saw-tooth wave, or simple or complex pulse, the parameters of which are adjustable in regard to amplitude, duration, repetition rate and other variables. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated as well as the outputs of various sensors which sense conditions prevailing in this substance whereby the user of the system can manually adjust it or have it automatically adjusted by feedback to provide an electrical stimulation signal of whatever type he wishes and the user can then observe the effect of this signal on a substance being treated.

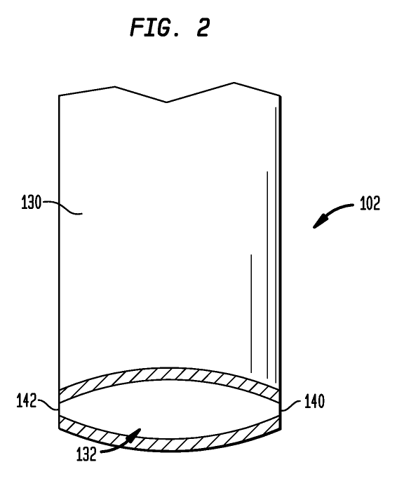

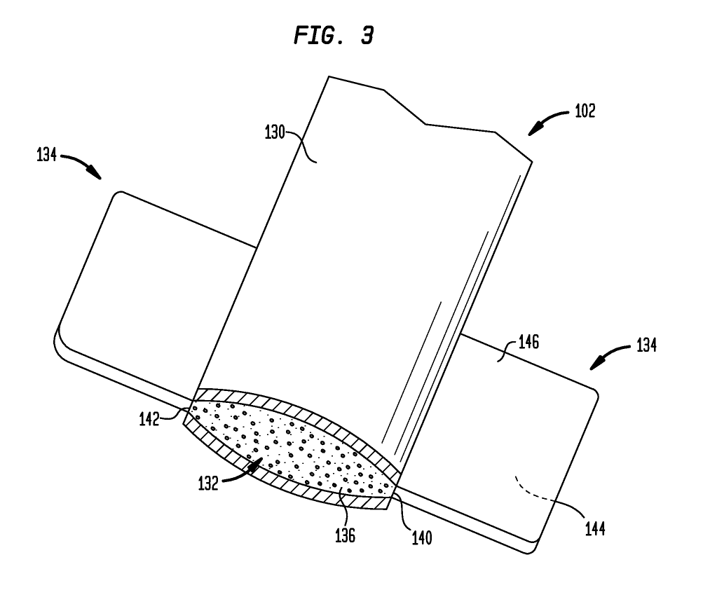

FIGS. 2 and 3 illustrate a distal end of electrode lead 102 according to the present invention. As shown, lead 102 comprises a shaft 130 having an internal lumen 132 with a fluid passage coupled to the source of fluid 106. The fluid used in the present invention may be any suitable fluid, such as air, saline or the like. An expandable electrode 134 is coupled to internal lumen 132 at the distal end of shaft 130. Electrode 134 includes an internal conductor (not shown) for coupling electrode 132 to connector 108 and electrical source 104. By way of example, the conductor may be a solid silver wire of about 0.25 mm diameter insulated with a PTFE material of about 0.33 mm diameter. The diameter of the insulating material of the conductor should be less than the internal diameter of lumen 132 such that fluid may freely flow therein despite the presence of the conductor. The conductor may be laser welded to the electrode 134 using known procedures.

Referring to FIG. 3, fluid 136 is delivered through internal lumen 132 of electrode lead 102, thereby expanding electrode 134 through lateral openings 140, 142 of electrode shaft 130. In this configuration, electrode 134 extends to either side of electrode shaft 130 such that a surface 144 of electrode 134 (shown on the underside of the electrode 134 in FIG. 3) is positioned in contact with or adjacent to the target region. In an exemplary embodiment, electrode 134 is insulated on surface 146 opposite surface 144 to avoid unnecessary stimulation to other structures within the epidural space. Alternatively, electrode lead 102 may comprise multiple electrodes with multiple fluid passages. For example, the device may include two separate electrodes that expand laterally outward in opposite directions from electrode shaft 130. In addition, it should be recognized that other configurations are possible. For example, electrode 134 may be positioned around the exterior of electrode shaft 130. In this embodiment, a fluid passage would extend along the exterior of shaft 130 and the electrode would be introduced into the patient in the deflated position.

Electrode 134 is specifically designed to contact or, be positioned in close proximity to, a substantially larger surface area of the dura than conventional spinal cord stimulation electrode(s). In one embodiment, electrode 134 is formed from a stretchable elastic material permitting the interior of the electrode to accommodate a variable volume of fluid ranging from a minimum deflated volume to a maximum inflated volume. The wall provides electrode 134 with an inflated shape within a body cavity or other target area within a patient approximating the shape of the body cavity or other target area. The elastic material of electrode 134 will preferably allow the outer wall of electrode 134 to conform to the tissue surrounding the body cavity or target area within the patient's body. Alternatively, electrode 134 may be designed to inflate to a pre-formed shape corresponding to a targeted area within a patient.

In the preferred embodiment, electrode 134 will extend laterally outward from shaft 130 by about 1 to 15 mm, preferably about 4-10 mm, and will have a length in the longitudinal direction of about 1 to 20 mm, preferably about 2 to 20 mm.

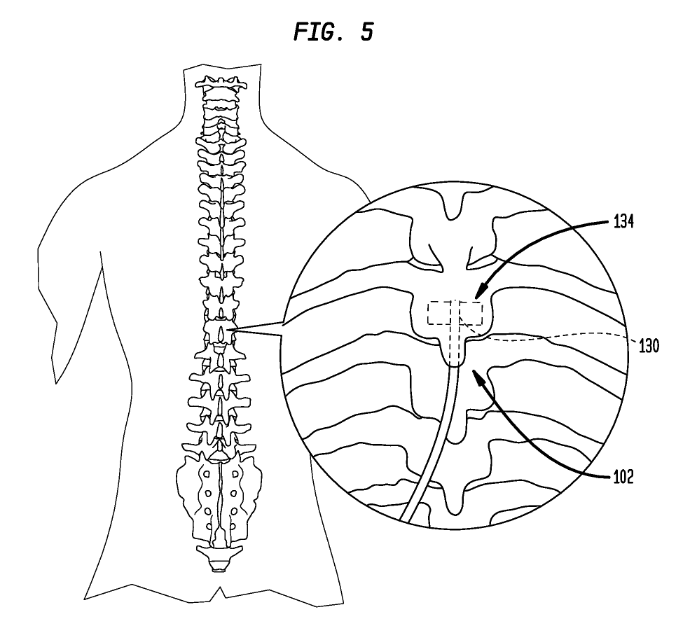

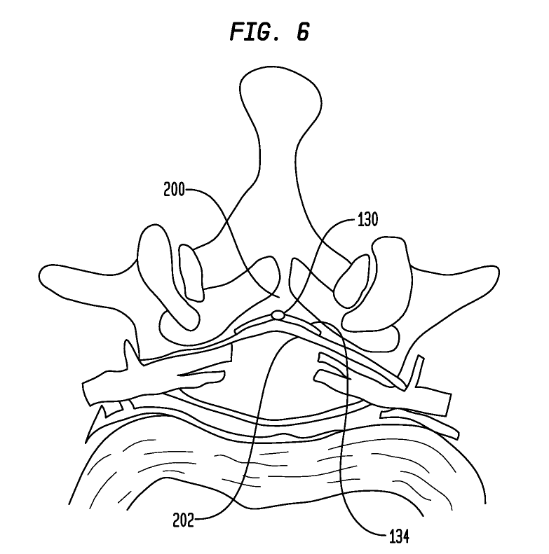

FIGS. 5 and 6 illustrate a method of modulating nerves within the epidural space 200 of a patient with the electrode lead 102 shown in FIGS. 2 and 3. In use, electrode lead 102 is introduced into the patient such that the distal region of electrode shaft 130 is adjacent to or in contact with a target area within the epidural space 200, such as the dura 202. The target area will of course vary depending on the application. In certain embodiments, the target area will be a nerve associated with a treatment location in the GI tract of a patient. The exact location of the target nerve in the spinal cord can be determined by first applying a detection electrical impulse to a selected target area in the spinal cord to create a sensation within the patient's GI tract. Depending on the desired location for treatment in the GI tract, the target area in the spinal cord can then be adjusted based on feedback from the patient. The adjustment may occur by physically moving the distal end 130 of electrode lead 102 or by alternating which electrodes of lead 102 receive the electrical impulse as is well known in the art. This feedback loop continues until the patient feels the sensation at the desired location in the GI tract.

In certain embodiments such as for treating post-operative ileus (described in more detail below), the target area will be between T5 to L2, preferably around T6 or T7. Electrode lead 102 may be introduced into the epidural space 200 percutaneously, via a laminectomy or in a variety of other manners well known in the art. In the exemplary embodiment, lead 102 is introduced percutaneously from a location below the target region (e.g., around L3 in the spinal column).

Once in position, fluid is delivered through fluid tube 112 and internal lumen 132 to the distal end of lead 102 to inflate electrode 134, thereby allowing the physician to position conductive surface 144 of electrode 134 adjacent to or in contact with the dura 202. The fluid may be any suitable biocompatible fluid, such as air or saline, although it is not expected that the fluid will leak through electrode 134 into the patient's body. An electrical impulse is then generated by signal source 104 and applied to electrode 134 to modulate nerves and/or muscles at the target region.

In certain embodiments for chronic use for treatment of pain, electrode lead 102 will be implanted within epidural space 200 and pulse generator 120 may be implanted, for example in the abdomen or buttocks, to apply electric impulse(s) to electrode 134. In such embodiments, the electrical impulse may be selected to block pain signals from reaching the brain such that the patient receives a mild tingling sensation in lieu of pain. In other embodiments such as treating post-operative ileus (described in detail below), electrode lead 102 may be used acutely for a period of time (e.g., from minutes to days) and then withdrawn from the patient (i.e., without permanently implanting lead 102 or pulse generator 120).

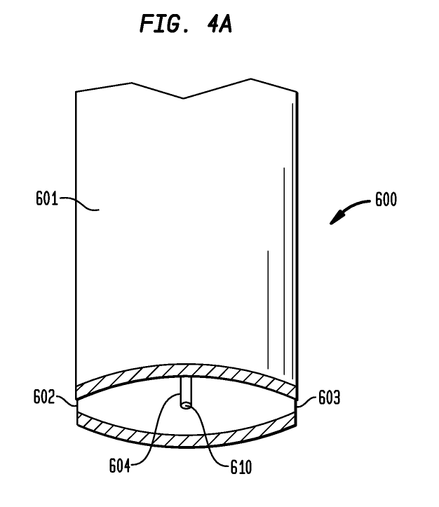

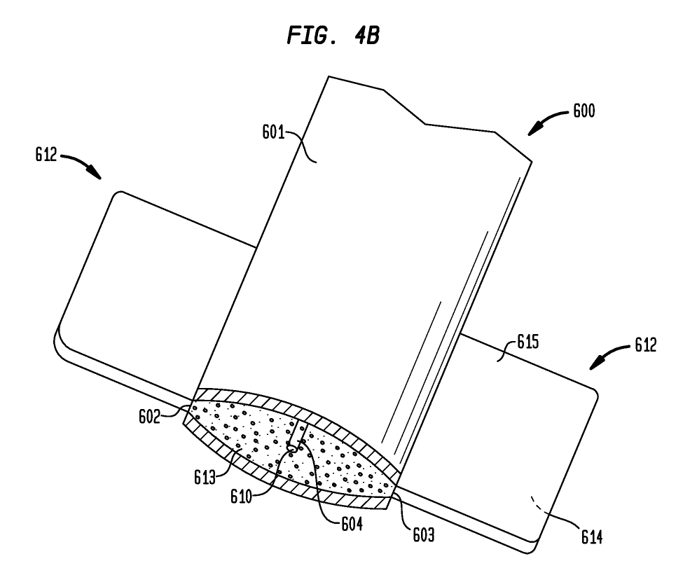

FIGS. 4A and 4B illustrate an alternative embodiment of the present invention. In this embodiment, the electrical impulse is passed from an internal electrode through electrically conductive fluid to, and through, an outer wall of a balloon. As shown, an electrode lead 600 includes a shaft 601 with an internal lumen 613 and an electrode 610 extending through lumen 613. A balloon 612 is coupled to the distal end of shaft 601 and designed to expand laterally outward through openings 602, 603 in shaft 601 in a similar manner as described above. A portion or all of surface 614 of balloon 612 is preferably formed of an electrically-permeable material, preferably a hydrophilic or ion-permeable material. By way of example, balloon 612 may be substantially formed from an ion-permeable, soft, flexible, and/or distensible material with a thickness of about 0.001 inches. Suitable balloon materials for use in the present invention include Pebax®, aromatic polyether polyurethane grades, such as Dureflex® from, for example, Deerfield Urethane in Whately, Mass., thermally conductive polymers or thermoplastic elastomers (TPE) such as those found at Cool Polymers, Inc. in Warwick, R.I. and the like. However, it will be recognized by those skilled in the art that a variety of commercially available balloon materials may be used to carry out the present invention.

Electrode 610 may be of a general cylindrical shape and may be formed from Pt-IR (90%/10%), although other materials or combinations or materials may be used, such as platinum, tungsten, gold, copper, palladium, silver or the like. Those skilled in the art will also recognize that a variety of different shapes and sizes of electrodes may be used. By way of example only, electrode shapes according to the present invention can include ball shapes, twizzle shapes, spring shapes, twisted metal shapes, annular, solid tube shapes or the like. Alternatively, the electrode(s) may comprise a plurality of filaments, rigid or flexible brush electrode(s), coiled electrode(s) or the like. Alternatively, the electrode may be formed by the use of formed wire (e.g., by drawing round wire through a shaping die) to form electrodes with a variety of cross-sectional shapes, such as square, rectangular, L or V shaped, or the like.

An electrically conductive fluid, preferably a saline solution, passes into the balloon 612 through lumen 613 to inflate same. The balloon 612 is sized, shaped and located about the electrode 610 such that when the balloon is inflated with fluid, the electrode 610 is substantially centrally located within an interior volume of the balloon 612. This configuration has several advantages over conventional electrode configurations, such as: (i) the metal of the electrode 610 is not too close to, and never comes in contact with, the patient's tissue, which means that there is no concern about tissue necrosis or excessive electric fields in the tissue; (ii) the electrode 610 may be used with direct current signal sources since any Faradic Products (e.g. OH⁻, H₂O₂) would not reach excessively high concentrations at the tissue site; (iii) as the balloon 612 is filled with saline, the surface of the balloon 612 wets and permits good contact with the surrounding tissue of the patient, which may otherwise be dry; and (iv) the material of the balloon 612 is preferably very soft and flexible such that it gently conforms to the surrounding tissue of the esophagus.

The electrical properties of the electrode 610, the fluid, and the material of the balloon 612 are preferably designed such that a resistance therethrough is no more than about 1000 Ohms, preferably no more than 500 Ohms and more preferably 200 Ohms or less. In an exemplary embodiment, the impedance through the electrode 610, the fluid, and the material of the balloon 122 should be no more than about 200 Ohms at 1000 Hz. The electrical properties of the fluid may be as important as those of the electrode 610 in this regard. The electrically conducting fluid should have a threshold conductivity to provide a suitable conductive path between electrode 610 and the outer wall of the balloon 612. The electrical conductivity of the fluid (in units of milliSiemans per centimeter or mS/cm) will typically be between about 1 mS/cm and 200 mS/cm and will usually be greater than 10 mS/cm, preferably will be greater than 20 mS/cm and more preferably greater than 50 mS/cm. In one embodiment, the electrically conductive fluid is isotonic saline, which has a conductivity of about 17 mS/cm. Applicant has found that a more conductive fluid, or one with a higher ionic concentration, will usually provide optimal results. For example, a saline solution with higher levels of sodium chloride than conventional saline (which is on the order of about 0.9% sodium chloride) e.g., on the order of greater than 1% or between about 3% and 20%, may be desirable. A fluid of about 5% saline (e.g., approximately 100 mS/cm) is believed to work well, although modifications to the concentration and the chemical make-up of the fluid may be determined through simple experimentation by skilled artisans.

As noted above, the material of the balloon 612 is preferably slightly water-permeable or hydrophilic so that when the balloon 612 is filled with saline, the surface of the balloon 612 wets. Preferably, when filled with 10 cc of saline, the flux of saline out of the balloon 102 (into a similar saline solution) should not exceed about 1 cc per hour. Lubrizol Tecophilic HP93A-100 is a material with these properties.

In an alternative embodiment, the electrode 610 may be implemented via the fluid itself within the balloon 612. Although a 5% saline solution would have a relatively high resistance compared to a metal electrode 610 implementation, those skilled in the art would appreciate that higher conductivity fluid solutions may be employed for such purposes or a larger diameter and/or shorter tube may be utilized to increase the conductivity. Additionally or alternatively, the conductor may be implemented using the conductive fluid used to fill the balloon 612; indeed, such fluid is within the passage anyway. Again, relatively high conductivity fluid would be desirable.

In another embodiment, the present invention may be used for treating gastrointestinal disorders, such as pain associated with IBS and/or gastric or intestinal motility disorders. In an exemplary embodiment, the present invention describes a method for reversing the temporary arrest of intestinal peristalsis as described more fully in commonly assigned U.S. patent application Ser. No. 12/246,605, which has already been incorporated herein by reference. Recent reviews in the art have discussed the potential application of electrical stimulation of the end organ, namely the stomach, small intestine or colon to improve motility. SCS may also be a useful treatment modality for dysmotility, particularly delayed gastric and intestinal motility following surgery.

Post-operative ileus (POI) is a common transient bowel dysmotility. POI is a frequent complication seen in a preponderance of major abdominal surgeries, as well as one of the most frequently encountered sequela of intra-peritoneal chemotherapy. The signs and symptoms associated with POI include abdominal pain and distension, reduced borborygmi, vomiting, nausea, early satiety, and an increased transit time for the passage of flatus and/or stool. POI frequently results in prolonged hospital stays as a consequence of gastrointestinal (GI) complications. Recent estimates of the medical costs incurred due to these complications exceed $1 billion annually. Clinical complications associated with POI include an increase in nasogastric tube reinsertion, intravenous volume maintenance and/or hydration, added nursing care, additional laboratory testing, increased re-admission, and more days in-hospital.

SCS may accelerate motility in patients with POI; following therapeutic SCS for chronic pain, patients report increased bowel movements and relief from severe constipation. Colonic motility has been assessed by others in two patients that underwent SCS for neurogenic bladder with concomitant severe constipation. Both received SCS at the level of the 8^{th} and 9^{th} thoracic vertebrae and reported spontaneous defecation within 12 hours and increased weekly bowel movements. In another case report, two patients received permanent spinal cord stimulator implants, where the generators were placed for pain management. While the adverse GI symptomatology varied between each patient, common to both was persistent diarrhea associated with stimulator use. These GI side effects were severe enough that both patients had the permanent stimulators removed in spite of excellent pain coverage. In contrast, a single case report found that SCS was able to eliminate diarrhea in a patient with irritable bowel syndrome (IBS), even after the beneficial effect on pain management abated. Taken together, these clinical reports in the art support a previously unconsidered association between SCS and alterations in gastrointestinal motility.

In this embodiment, an electrode device as described above is introduced into the patient and placed in contact with, or close proximity to, at least one of the celiac ganglia, cervical ganglia and thoracic ganglia of the sympathetic nerve chain. An electric signal is applied to the electrode and/or balloon to induce at least one of an electric current, an electric field and an electromagnetic field in the sympathetic nerve chain to modulate and/or block inhibitory nerve signals thereof such that intestinal peristalsis function is at least partially improved. Alternatively or additionally, the electric current, electric field and/or electromagnetic field may be applied to at least a portion of the splanchnic nerves of the sympathetic nerve chain, and/or the spinal levels from T5 to L2.

The electrode may be introduced into the epidural space of the patient after the surgery has been completed. As described more fully above, the electrode is preferably introduced through a small portal and then expanded inside the epidural space to achieve a larger footprint of contact on the dura. This ensures that the electric impulse will target the selected nerves to sufficiently influence the therapeutic result. In addition, it inhibits migration of the electrode within the epidural space and provides for a more efficient and effective treatment.

As described more fully in the Ser. No. 12/246,605 patent application, drive signals may be applied to the one or more electrodes to produce the at least one impulse and induce the current and/or field(s). The drive signals may include at least one of sine waves, square waves, triangle waves, exponential waves, and complex impulses. The drive signals inducing the current and/or fields preferably have a frequency, an amplitude, a duty cycle, a pulse width, a pulse shape, etc. selected to influence the therapeutic result, namely modulating some or all of the nerve transmissions in the sympathetic nerve chain. By way of example, the parameters of the drive signal may include a square wave profile having a frequency of about 10 Hz or greater, such as between about 15 Hz to 200 Hz, and more preferably between about 15 Hz to about 50 Hz. The drive signal may include a duty cycle of between about 1 to 100%. The drive signal may have a pulse width selected to influence the therapeutic result, such as about 20 us or greater, such as about 20 us to about 1000 us. The drive signal may have a peak voltage amplitude selected to influence the therapeutic result, such as about 0.2 volts or greater, such as about 0.2 volts to about 20 volts.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A device for delivering electrical energy to a patient, comprising:
an enclosure having a longitudinal axis and an outer wall being movable between a collapsed configuration for introduction into the patient wherein the outer wall extends laterally outward from the longitudinal axis by a first distance and an expanded configuration for treating the patient wherein the outer wall extends laterally outward from the longitudinal axis by a second distance, wherein the second distance is greater than the first distance;
an electrode coupled to at least a portion of the outer wall of the enclosure; and
a source of electrical energy coupled to the electrode for delivering an electrical impulse to the electrode in the expanded configuration.

2. The device of claim 1 wherein the enclosure comprises an outer wall defining an interior, the device further comprising a fluid passage coupled to the interior of the electrode and a source of fluid for delivering a fluid into the interior of the enclosure and expanding the outer wall into the expanded configuration.

3. The device of claim 1 wherein the outer wall of the enclosure has a larger surface area for contacting tissue in the expanded configuration.

4. The device of claim 1 wherein the outer wall of the enclosure is the electrode.

5. The device of claim 1 wherein a portion of the outer wall of the enclosure is the electrode.

6. The device of claim 1 wherein the electrode is housed within the enclosure and electrically coupled to the outer wall.

7. The device of claim 6 wherein the electrode is spaced from the outer wall and electrically coupled to the outer wall by an electrically conductive fluid.

8. The device of claim 1 wherein the outer wall expands laterally outward in first and second directions from the longitudinal axis in the expanded configuration.

9. The device of claim 8 wherein the outer wall forms a substantially planar surface extending in opposite directions from the longitudinal axis in the expanded configuration.

10. The device of claim 1 wherein the outer wall forms first and second contact surfaces extending laterally outward from the longitudinal axis in the expanded configuration, wherein the electrode forms part of the first contact surface and the second contact surface comprises an insulating material.

11. The device of claim 10 wherein the first and second contact surfaces are on opposite sides of the outer wall in the expanded configuration.

12. The device of claim 1 wherein the enclosure and the electrode are sized and shaped in the collapsed configuration for a percutaneous penetration into an epidural space of a patient.

13. The device of claim 1 wherein the source of electrical energy is an electrical signal generator operating to apply at least one electrical signal to the electrode such that, when the enclosure is positioned in a spinal cord of the patient, an electro-magnetic field emanates from the electrode to at least one of nerves and muscles in a vicinity of the spinal cord.

14. The device of claim 13 wherein the electrical signal generator operates such that the at least one electrical signal is of a frequency between about 1 Hz to 3000 Hz, a pulse duration of between about 10-1000 us, and an amplitude of between about 1-20 volts.

15. A method for treating an ailment in a patient comprising:
introducing an enclosure through a percutaneous penetration of a patient to a target location adjacent to or near a spine of the patient, the enclosure having a longitudinal axis;
expanding an outer wall of the enclosure in a lateral direction from the longitudinal axis;
contacting a tissue of the patient at the target location with at least a portion of the outer wall; and
applying electrical energy to an electrode coupled to the portion of the outer wall to modulate one or more nerves within the spine of the patient.

16. The method of claim 15 wherein the expanding step is carried out by delivering a fluid into the enclosure.

17. The method of claim 15 wherein at least a portion of the outer wall defines the electrode.

18. The method of claim 15 wherein the electrode is housed within the enclosure and spaced from the outer wall, the method further comprising coupling the electrode to the outer wall by delivering an electrically conductive fluid into an interior of the enclosure.

19. The method of claim 15 wherein the expanding step comprises expanding the outer wall in at least two directions from the lateral axis.

20. The method of claim 15 wherein the expanding step comprises forming a substantially planar contact surface with the outer wall of the enclosure, the planar contact surface having a larger surface area than a diameter of the percutaneous penetration in the patient.

21. The method of claim 20 wherein at least a portion of the substantially planar contact surface comprises the electrode.

22. The method of claim 20 further comprising insulating a surface of the outer wall opposite the substantially planar contact surface.

23. The method of claim 15 wherein the applying step is carried out by applying at least one electrical impulse to the electrode such that an electro-magnetic field emanates from the electrode to at least one of nerves and muscles within the spine of the patient.

24. The method of claim 15, further comprising contacting a return electrode to an external portion of the patient and emanating an electro-magnetic field from the electrode through the tissue in a substantially radial pattern.

25. The method of claim 15 wherein the applying step comprises applying an electrical impulse to a sympathetic nerve chain of a patient to modulate nerve signals thereof such that intestinal peristalsis function within the patient is at least partially improved.

26. The method of claim 15 wherein the applying step is carried out by applying an electrical signal to the electrode of a frequency between about 10 Hz to 200 Hz, a pulse duration of between about 20-400 us, and an amplitude of between about 1-20 volts.

27. The method of claim 26 wherein the frequency is between about 25 to 50 Hz.

28. The method of claim 15 wherein the applying step is carried out by applying an electrical impulse to the electrode sufficient to increase an intestinal motility of the patient.

29. The method of claim 15 wherein the applying step is carried out by applying an electrical impulse to the electrode sufficient to increase a gastric motility of the patient.

30. The method of claim 15 wherein the applying step is carried out by applying an electrical impulse to the electrode sufficient to treat pain.

31. The method of claim 30 wherein the pain is visceral pain associated with irritable bowel syndrome.

32. A method for treating a temporary arrest of intestinal peristalsis in a patient comprising:
introducing an electrode to a target location adjacent to or near a spine of the patient;
expanding the electrode in a lateral direction relative to a longitudinal axis of the electrode; and
applying an electrical impulse to the electrode sufficient to increase intestinal motility of the patient.

33. The method of claim 32 wherein the electrical impulse has a frequency of about 25 Hz to 50 Hz, a pulse width of about 100 to 1000 us and an amplitude of about 0.2 to 20 volts.

34. The method of claim 32 wherein the introducing step comprises advancing the electrode through a percutaneous penetration in the patient and wherein the electrode is expanded in a lateral direction relative to the percutaneous penetration.

35. The method of claim 32 wherein the electrical impulse is sufficient to modulate one or more sympathetic nerves in the patient.
